(19) United States
(12) Patent Application Publication
(10) Pub. No.: US 2008/0255554 A1
(43) Pub. Date: Oct. 16, 2008

(54) External Fixator for Osteosynthesis or Bone Gap Manipulation

(76) Inventors: Jens Richter, Basel (CH); Andre Plass, Zuerich (CH); Achim Haeussler, Wetzikon (CH); Juerg Gruenenfelder, Zuerich (CH); Gregor Zuend, Herrlberg (CH); Marko Turina, Zuerich (CH)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 10/594,854

(22) PCT Filed: Mar. 30, 2005
(86) PCT No.: PCT/IB2005/000826

§ 371 Date: Jun. 27, 2008

(30) Foreign Application Priority Data
Apr. 1, 2004 (IB) PCTIB2004001008

(51) Int. Cl. A61B 17/03; A61B 17/00

(52) U.S. Cl. 606/57; 606/54

(57) ABSTRACT

The invention relates to an external fixator which has a retaining member (1) and connecting elements (2) which can be connected without screws to a bone or bone parts (5), in particular to both halves of an opened sternum, and provide possibilities for increasing or reducing the compressive stress between the two bone parts or sternum halves.

This application is a national stage application of PCT/IB2005/000826, filed Mar. 30, 2005 which claims priority to PCT/IB2004/001008, filed Apr. 1, 2004.

### FIELD OF THE INVENTION

The invention relates to an external fixator for osteosynthesis or bone gap manipulation, comprising an external retaining member and connecting elements with retaining ends held in the retaining member and with contact ends to be placed on the bone for connecting the retaining member to the bone or to bone parts. The invention relates in particular to an external fixator for closing a dislocated bone and the parts thereof, for example a sternum which has been cut through.

### BACKGROUND OF THE INVENTION

Document IL-A-122418/2, dated Feb. 12, 1997, describes such an external fixator which consists of an external rod as a retaining member, to which threaded rods are screwed as connecting elements. There are two screwable plates on each of the contact ends of the threaded rods, one of which comes to rest below the sternum and one above the sternum. In the position of use, a sternum is sandwiched between the plates. Consequently, the relative distance between sternum and retaining member is variable. The threaded rods lie laterally next to the sternum. By moving the threaded rods on the retaining member, more or less pressure can be applied to the sternum from the lateral to the medial direction. A reduction of the pressure to a negative force is not possible since the plates and threaded rods would migrate away from the sternum if the threaded rods were moved laterally (cf. FIG. 6 of document IL-A-122418/2).

In this configuration, another detrimental effect occurs when the pressure is increased: the threaded rods, which rest laterally against the sternum, bend in the lateral direction so that there is a tendency for the lower plate to swivel outwards and the upper plate inwards (in a medial and downward direction). This leads to an increase in the pressure on the upper region of the gap in the sternum and to a reduction of the pressure in the lower region of the gap. This can lead to undesired deformations of the sternum.

Furthermore, it is disadvantageous if the lower plate comes to rest over a relatively large area below the sternum, i.e. in a critical region close to the heart. This is undesirable for heart surgery and cardiology.

On the other hand, this configuration permits the removal of the fixator after healing of the bone gap without a further operation, by virtue of the fact that the plates become loose and the threaded rods can then be extracted in an upward direction. Even when the plates have not yet become loose, the threaded rods can be removed from the plates by turning.

Compared with other known methods, this is advantageous since in this method parts of the fixator remain in the patient, in particular wires or plates.

Systems which remain in the patient have been published in the following documents: WO88/06022; U.S. Pat. No. 5,318,566; U.S. Pat. No. 4,583,541; U.S. Pat. No. 6,217,580; U.S. Pat. No. 6,540,769 and DE U 20309158. As a rule these systems use the traditional method of winding wires around a sternum and twisting these wires.

Other conventional external fixators in which bone screws are the connecting elements have the disadvantage that it is necessary to drill into the bone. This is problematic when the bones are porous or thin. In addition, there is a high degree of uncertainty when drilling a sternum since medistinal perforation of the sternum should be avoided. Moreover, when a bone screw is improperly mounted, it can penetrate into the space below the sternum.

### SUMMARY OF THE INVENTION

The object of the invention is the development of an osteosynthetic connection system which permits simple, rapid and safe handling in the region of the sternum, particularly in cases of heart surgery. Because of its location, the system is subject to very high intermittent forces from concomitant thoracic movement if the patient experiences strong inhalation or exhalation or is coughing. The anchoring of the system presents another problem. The sternum is a flat bone which may additionally exhibit osteoporotic changes. The anchoring may be torn out, and there is also the danger of pleural/pericardial injury due to bone perforation. Because little subcutaneous tissue is present in the region of the sternum, wound healing should be critically assessed. Therefore, any additional burden on the soft tissue should be avoided by use of an external fixator. Materials which are well tolerated by tissue should be used.

Some definitions are helpful in the understanding of this invention. “External” means that substantial parts of the fixator, specifically the retaining member, comes to rest outside a patient's body in the application. Parts of the fixator, in particular the connecting elements, naturally engage the bone. However, they do not penetrate into the bone. In addition to the connecting elements, it is also possible to use wires, ropes or cables which may optionally penetrate the bone—as known in the art. “Connecting elements” are generally elongated components which come to rest between the retaining member and the bone and mutually support the two parts. “Dislocated bone” means all bones which have been divided into bone parts, by fracture or intervention, and in particular bones which have been split in their longitudinal direction, such as a sternum after a heart operation.

The external fixator should allow individual friction adjustment. In this invention, there is an initial compression at the bone gap, which can be changed into a tension at the bone gap if necessary for improved bone healing. This is not possible in the case of all conventional methods, with the exception of a conventional external fixator with bone screws.

However, external systems with bone screws often cannot be used precisely on a sternum.

The system must be worn over a period of at least two months. Although it may be more cumberson for the patent to wear than internal wires, it is much more convenient to remove because no operation is required.

Ideally, the device would be scarcely noticed by the patient while giving him a safe feeling. The basic requirements are a flat design, low weight and simple hygienic handling.

This object is achieved by the design of the invention and its special embodiments. By an external fixator for osteosynthesis or bone gap manipulation, which is equipped, in a manner known in the art, with an external retaining member and connecting elements with retaining ends held in the retaining member and. Contact ends are placed on the bone for connecting the retaining member to the bone or to bone parts, unlike traditional fixators that screw into the bone. This is a major advantage, particularly for small and thin bones and porous bones. The contact ends are supported only on the surface of the bone and are formed in such a way that the contact ends or connecting elements exert a lateral clamping/compression pressure on the bones or the bone parts. On a sternum, for example, this would be a lateral pressure in the direction of the sagittal plane.

Furthermore, according to the invention, a control element supported in the retaining member is coordinated with each connecting element in the retaining member, so that the clamping pressure can be varied.

As a result of this design, it is possible, to manipulate the gap between the bones in a controlled manner without having to drill into the bone, i.e. to increase or to decrease it, or to apply, to increase or to reduce a closing pressure on the gap.

In order also to be able to keep the contact ends pressed against the bones from the distal direction, various possibilities—depending on the type of bone—are conceivable in the context of the invention. Thus, a fixator according to the invention may be kept pressed against the bones by a suitable bandage.

According to a particular embodiment of the invention, however, such contact pressure is not necessary since, the contact ends are formed in a spoon-like manner so that they can at least partly surround a bone from the lateral direction. The extent of this surrounding depends on the type of bone and on its formation. For proper spoon-like formation, the connecting elements and hence the retaining member must be held by themselves on the bone.

It is advantageous if the contact ends are profiled so that they are held without slipping when used on the bone surface. The use of a counter-holder connected to the retaining member dispenses with the need for bandages or spoon-like formations because the counter-holder would ensure the connection between bone and connecting elements. However,in this embodiment (with surrounding alone), it is scarcely possible to exert a negative pressure on the bone gap. But variation of the pressure from low to high is still readily possible.

The counter-holder preferably comprises at least one flexible, wire- or cable-like loop which can be wrapped around the surface of the bone and can be fixed indirectly or directly to the retaining member. Thus, a surgical material known in the art, such as a wire or a cable, is used. This performs the function not only of connecting the bone parts but also of keeping the external fixator pressed against the bone.

An embodiment with such loops exists, for example, when the loop can be wrapped along the spoon-like formations and around the surface of the bone. This results in a bone-protecting path since it is precisely in the edge region of bones, especially in flat bones, that the localized load due to wires or cables is particularly great. On the other hand, there are applications in which such spoon-like surrounding systems tend to be troublesome; for example, in the case of the sternum, it is not desirable to have hard inflexible parts, since the heart beats directly below and should be irritated as little as possible after surgery.

For this reason, variants without surrounding systems are preferred for this invention. In such or other variants, the connecting elements are, U shaped or I shaped in section, so that the loop can be led in a U or in an I, and that, in the assembled state, each loop wraps around one connecting element each on a bone part and one connecting element each on the other bone part and can fix the two bone parts on the retaining member. In a preferred embodiment, the connecting elements are tubular and can cooperate with the loop or the loops so that, in the assembled state, each loop winds through one connecting element each on a bone part and another connecting element each on another bone part and wraps around both bone parts and can be fixed on the retaining member.

The design of the connecting element end which faces the bone (contact end) is such that the bone parts can be subjected to pressure from the lateral direction as well as in the lateral direction so that any desired bone gap manipulation is possible. This design was successfully tested on a prototype. It is distinguished by a compact appearance and protects the loop from the environment.

In a further development of this embodiment, the tubular connecting element has at least one lateral orifice for the entrance or emergence of the loop that is a distance away from the contact end. This design is advantageous because the contact end can rest against the bone undisturbed by the cable. In fact, the cable leaves the connecting part above the contact end. However, other conceivable embodiment exist in which the contact end has, in its end face, a slot which receives the loop or the cable when it leaves the tube.

Since the fixator tends to be bulky, it might reduce the X-ray visibility of body parts underneath, such as the heart and lungs. In order to prevent this, a particular embodiment ensures that the connecting elements and/or the retaining member are composed of X ray-transparent material which consists of light metal or of a light metal alloy or of carbon or of a carbon-reinforced material. It is also advantageous if the cable and the loops consist of carbon, since this material is well tolerated and is inert to many body substances and therefore does not intergrow with a wound.

Particular embodiments of the contact end are possible if the contact end is bevelled in the manner of a wedge or rounded along a curve and preferably toothed. Depending on requirements, optimum support can thus be chosen. It is particularly preferable if the connecting elements can be chosen from a group of different embodiments and can be inserted into a retaining member so that the ideal connecting element can be chosen for the specific point of use.

Clamping screws—preferably locking screws—serve for fastening the cable in or on the retaining member. Locking screws are clamping screws which have a built-in latch mechanism or the like, so that clamping is possible but release of the clamp is achieved only by special measures (e.g. latch release). As a result of this preferred development, a clamped cable or a clamped loop can thus be retensioned in a simple manner turn by turn without having to fear an accidental decline in the tension.

A particular universality of the design is achieved thereby if the two connecting elements, coordinated in each case with one another in a parallel transverse plane or in a normal plane relative to the retaining member, are displaceably or preferably pivotably mounted therein transversely to the longitudinal dimension thereof. This universality increases even further if the connecting elements are additionally displaceable or pivotable in one plane each parallel to the sagittal plane or in one plane each normal to the retaining member, along the longitudinal dimension thereof. Thus, each connecting element can be adjusted in two planes, and the adjustability in the transverse planes serves for tensioning the loops and at the same time for subjecting the bone parts to lateral displacement loads. This design permits gentle, nondestructive osteosynthesis in an optimum manner, making it possible to remove the external fixator completely when the osteosynthesis is complete, without exogenous materials remaining in the body. The loops can be pulled out in the proximal direction in the same way as sutures, as can the connecting elements. The retaining member itself is always outside the body.

Since the cable rests against the bone along a line, there is no localized load, as is otherwise the case with screwed systems. Since the cable is flexible or elastic, this clamping method corresponds fairly well to the bone physiology. This is advantageous because the bone is kept optimally pressed against the connecting elements and the latter can therefore display their bone gap manipulation effect—which is adjustable by the control elements. Actuation of the control elements therefore not only leads to an adjustment of the control elements but simultaneously also affects the tension of the loop, thus permitting easy operation.

The invention also comprises embodiments in which the loop tension is adjustable independently of the connecting element adjustment. The invention also comprises attachments in which, in the assembled state, the loops are led through thin holes in the bone instead of being led laterally past the bone. This may be advantageous where it is desired to secure the position of the loops in the bone to prevent slipping.

A control element comprises a screw or a threaded pin, which comes into contact indirectly or directly from the lateral direction with one connecting element each and thus defines its pivot or displacement position relative to the retaining member.

Restoring elements, such as springs or springy support parts, can also be provided for such connecting elements, in order to be able to operate a fixator according to the invention in as defined during surgery.

In connection with the invention, a zip system for skin closure can be provided when the fixator has been inserted, said system being perforated in the region of the connecting elements for passage of the latter.

For improved hygiene, it is possible to ensure that the connecting elements comprise an elastic closure which—in the case of tubular connecting elements—closes its cavity but can be passed through the loop The closure is preferably comprised of a sterile or biocidal material, such as wax, fabric or foam.

Either in combination with the closure, or independently thereof, the retaining member may comprise a cover which makes it possible to close the region of the retaining ends, for further improvement of the hygiene. This fixator is therefore a completely self-contained system without an open connection to the outside.

The loop can be fixed to the retaining member or can be held in a clamping mechanism which can be released or clamped stepwise or in stages. A locking screw is preferred for this too; however, an attachment similar to a gear or in the form of a tension lock can be provided. For example, this can also comprise a screw nipple of the Bowden cable type.

The particularly preferred embodiments of the invention for a closure of a sternum opened along the sagittal plane comprise an external retaining member and connecting elements with retaining ends held in the retaining member and with contact ends to be placed on the bone. The contact ends connect the retaining member to the bone or to bone parts, and a counter-holder holds the bone distally in the direction of the retaining member. The connecting elements or the contact ends are screws or the like and the contact ends are only supported on the surface of the bone and are formed in such a way that they, or the connecting elements, can exert a lateral clamping pressure (directed towards the sagittal plane) on the bone or the bone parts. The counter-holder comprises at least one flexible, wire-like or cable-like loop which can be wrapped around the medial and lateral surface of the bone and can be fixed indirectly or directly to the retaining member.

As already described, it is preferable in this embodiment if a control element, which is supported in the retaining member and by means of which the clamping pressure of the loop can be varied, is coordinated with each connecting element or each loop. Each loop should be supported on and/or led to at least two connecting elements each.

In the invention, carbon fibre cables are preferably used. These must have a certain degree of flexibility. On the one hand, they must permit problem-free introduction and removal by being smooth enough to slide around the sternum with a certain degree of bending. On the other hand, they must have a high degree of tensile strength.

The carbon fibre material is well tolerated by the skin, and moreover substantial intergrowths with the surrounding tissue are kept at a low level, which is important due to the duration for which it is worn (at least 2 months). The carbon fibre cables are positioned intercostally.

The cables pass through tubular connecting elements which are introduced transcutaneously, rest on the sternum and do not penetrate the latter.

For the implantation, incisions of about 1 to 1.5 cm from the wound edge are made and are widened by a hollow pin. The cables, which may run in two possible ways (see below), are then led through this hollow pin and are threaded into the connecting elements or into the retaining member, which is in the form of a plate. The connecting elements are then passed through the skin, sliding on the carbon fibre cables, and pressed on the sternum, depending on the model. Owing to their profiled design, they are well retained there.

The plate itself comprises a material which is not opaque to X rays, such as plastic, hard rubber, carbon, light metal or the like.

### BRIEF DESCRIPTION OF THE DRAWINGS

The invention is explained in more detail by way of example with reference to the figures. The figures are described in relation to one another and overall. Identical reference numerals denote identical components, and reference numerals having different indices indicate functionally identical components.

FIG. 1 shows the schematic overall design with cross-loop;

FIG. 2 shows a schematic diagram of an alternative attachment with single loop;

FIG. 3 shows an adjustment of the attachment shown in FIG. 2 and

FIG. 4 shows a skin closure system which can preferably be used with the invention.

Two models of an external fixator according to the invention, having different cable paths and methods of fixing, are presented:

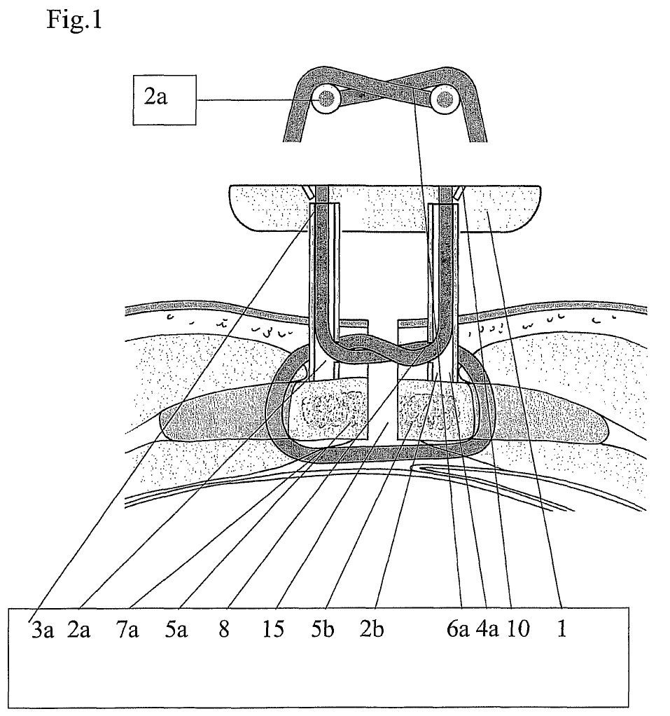

FIG. 1 shows an attachment with cross-loop 7a:

In the lower part, a section along the transverse plane through a patient, in particular through the sternum, with inserted external fixator is shown. A section along a frontal plane is shown above this, where only loop 7a simultaneously exerts the control tension on connecting elements 2a and 2b and thus applies more or less pressure to a bone gap 15.

The loop 7a is placed between connecting element 2a, bone part 5a and connecting element 2b. Second bone part 5b is introduced basally and medially. In the assembled state, said loop therefore consists of a crossed carbon fibre cable threaded into the hollow connecting elements 2a and 2b, three or four of which are used per bone-part 5a or 5b. The retaining ends 3a and 3b of the connecting elements are fastened to plate-like retaining member 1 with a stable angle, and contact ends 4a and 4b of the connecting elements 2a and 2b, respectively, are anchored in the sternum in the manner of “ski stocks” but do not penetrate into the bone 5. The carbon fibre cables 7a are individually fixed in the retaining member 1 by graduated screws or by screws or threaded pins 10.

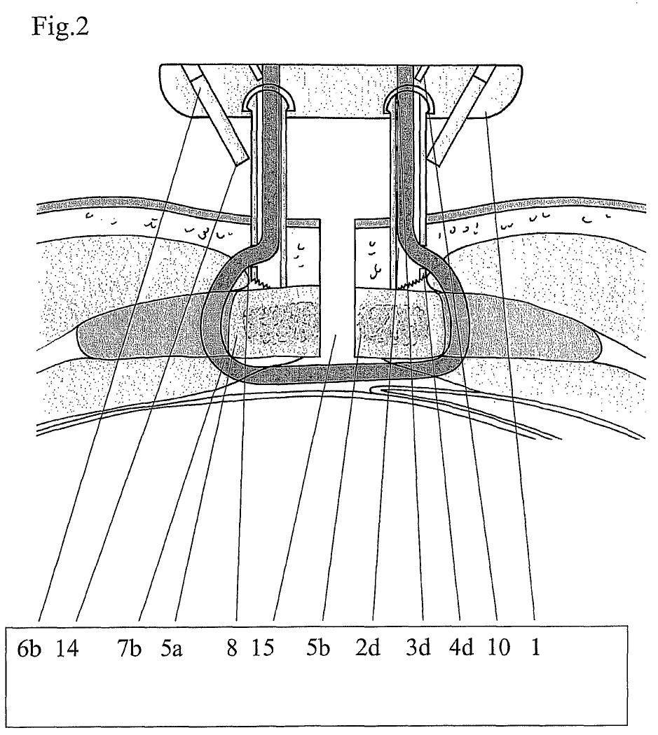

In contrast to FIG. 1, FIG. 2 shows only one one-part diagram with a single loop of the loop 7b and an articulated system of retaining ends 3c and 3d in the retaining member 1. The articulated system is formed in such a way that the connecting elements 2c and 2d can be pretensioned by means of control elements 6b which, with the aid of a clamping mechanism 14, make contact laterally with the connecting elements 2c and 2d. The clamping mechanism 14 is only indicated since the person skilled in the art can devise a very wide variety for this purpose, in particular those where the connecting elements can be subjected not only to pressure but also to tension. Springs which cooperate with the clamping mechanism 14 so that the connecting elements 2 are each subjected to pressure can also be provided for this purpose. In the case of the attachment according to FIG. 2, lateral introduction of the carbon fibre cables 7b into the base of the hollow connecting elements 7c,d and fixation in the retaining member 1 by means of graduated screw 10 are effected. There is no cross-over of the cables 7b. In order to generate compressive force at the upper edge of the sternum 5a and 5b, a lateral pressure on the connecting elements 2c and 2d and hence also on the cables 7b running therein is generated by the clamping mechanism 14. This is possible by various types of techniques, three of which are mentioned by way of example:

a) Worm thread

b) Screw thrust system, fastening of the pin to the plate by means of a barrel joint

c) Screw tension system, connecting elements run in a control groove of a cam which can be rotated by means of screw force.

In the case of all techniques used, a basal one-point fixation in the oblique connecting element position is advantageous.

FIG. 2 shows the system after assembly but without strong compression.

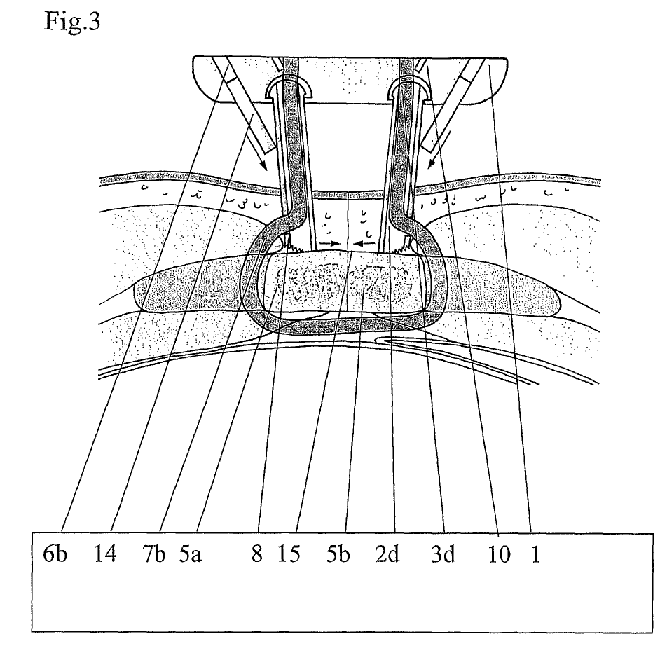

In FIG. 3, the connecting elements 2c and 2d are swivelled inwards under the pressure of the clamping mechanisms 14 and control elements 6b acting laterally on them, and the two bone parts 5a, 5b of the sternum are therefore compressed in the medial direction, which leads to closure of the gap 15.

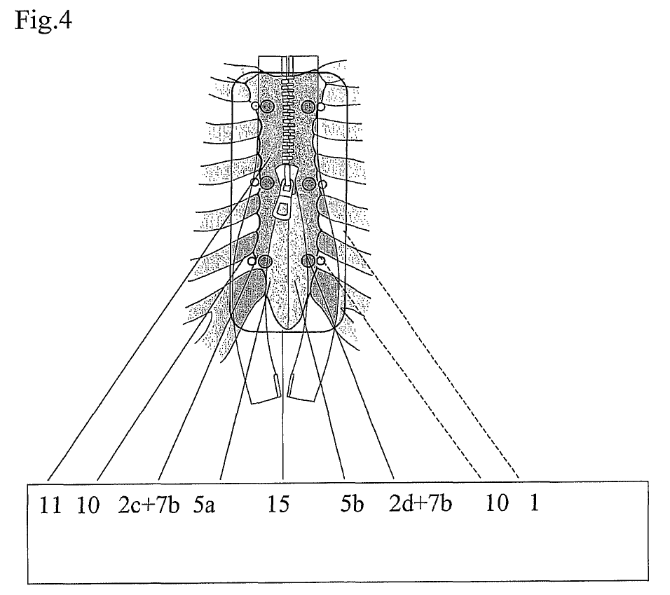

The skin closure should not be effected in the customary manner by means of an intracutaneous suture. The use of a zip system 11, as shown in FIG. 4, is recommended. It can easily be applied and removed. In addition, it has the major advantage of simple opening of the zip 11 with subsequent checking of the wound without having to remove the entire fixator beforehand, if problems with wound healing should be encountered.

### Advantages of the Preferred Embodiment

Forces are distributed throughout the system by the plate-like retaining member 1 located about 1 to 2 cm above skin level, so that the compressive force at the base of the connecting elements 2c, 2d decreases in the direction of the sternum (at the contact ends 4c, 4d), and the risk of perforation is minimized in this manner. This is beneficial for patients with osteoporosis.

With a cover (not shown) and/or application of an insert to the hollow connecting elements 2 (likewise not shown), a completely self-contained system results, which permits no direct access to the mediastinum for pathogens.

No intergrowths owing to the carbon fibre material, it being possible for the connecting elements 2 as well as the retaining member I to be composed of carbon. Individual friction adjustment by graduated screws. Size and height flexibility is achieved through exchangeable connecting elements 2.

Flexibility of respiration through certain elasticity in the entire system, particularly in loop 7.

Designable, exchangeable retaining members in the form of a plate

Improved hygiene

Flat design

The invention therefore relates primarily to an external fixator which has a retaining member 1 and connecting elements 2 which can be connected without screws to a bone or bone parts 5, in particular to both halves of an opened sternum, and provide possibilities for increasing or reducing the compressive stress between the two bone parts 5 or sternum halves.

While reference is made to a system for a sternum; the invention is not restricted thereto but rather is also available for other bones.

The list of reference symbols and the drawing, together with the subjects described or protected in the claims, are an integral part of the disclosure of this Application.

### LIST OF REFERENCE NUMERALS

1—Retaining member . . .
2a,b,c,d—Connecting element, U-shaped, I-shaped, tubular . . .
3a,b,c,d—Retaining end . . .
4a,b,c,d—Contact end, bevelled, formed along a curve . . .
5a,b—Bones, bone parts . . .
6a,b—Control element, in the case of FIG. 1: loop 7a
7a,b—Counter holder, loop . . .
8—Hole
9—Normal plane
10—Screw or threaded pin or locking screw
11—Zip system
12—Region of the connecting elements
13—Closure
14—Clamping mechanism
15—Bone gap

1-23. (canceled)

24. An external fixator for osteosynthesis, comprising:
an external retaining member configured and dimensioned for bridging separated bone segments;
a plurality of connecting elements configured and adapted for applying a clamping force to the separated bone segments, each connecting element having a first end and a second contact end, the first end configured and adapted for attachment to the retaining member and the second contact end configured and adapted for surface attachment to one of the separated bone segments without penetrating the bone segment; and
a plurality of control elements configured and adapted for varying the clamping pressure applied by the connecting elements, at least one control element being coordinated with a connecting element and supported in the retaining member.

25. The fixator of claim 24, wherein the contact ends are spoon-shaped.

26. The fixator of claim 24, wherein the contact ends are beveled and toothed.

27. The fixator of claim 24, further comprising a counter-holder, the counter-holder connected to the retaining member and including at least one flexible loop of sufficient length to wrap around a medial and lateral surface of at least one bone segment.

28. The fixator of claim 27, wherein the loop, the connecting elements and the retaining member are composed of X-ray-transparent material.

29. The fixator of claim 27, wherein the loop, the connecting elements and the retaining member are composed of a material selected from the group consisting of light metals, light metal alloys, carbon and carbon-reinforced materials.

30. The fixator of claim 27, wherein the at least one loop is coordinated with two connecting elements, and is supported by the connecting elements.

31. The fixator of claim 27, wherein the connecting elements are U-shaped and each loop is wrapped around one connecting element

32. The fixator of claim 27, wherein the connecting elements are tubular, and each loop winds through one connecting element on one bone part and another connecting element on another bone part.

33. The fixator of claim 24, wherein the connecting element has at least one lateral orifice a distance away from the contact end.

34. The fixator of claim 27, wherein clamping screws fix the loop to the retaining member.

35. The fixator of claim 24, wherein the control elements include screws that laterally contact at least one connecting element and pivot relative to the retaining member.

36. The fixator of claim 24 wherein a skin closure zip system is located in the region of the connecting elements.

37. The fixator of claim 24, wherein the retaining member forms a cover forming a completely self-contained system.

38. The fixator of claim 27, wherein the loop is held in an adjustable clamping mechanism.

39. The fixator of claim 38, wherein the clamping mechanism comprises a screw nipple of the Bowden cable type.

40. A method for osteosynthesis using an external fixator, comprising:
attaching connecting elements to a bone surface, the connecting elements having contact ends which are supported on the bone surface without penetrating the bone surface;
connecting the connecting elements to an external retaining member by means of retaining ends which are held in the retaining member;
holding the bone distally in the direction of the retaining member by means of a counter-holder, the counter-holder comprising at least one flexible loop which wraps around a medial and lateral surface of the bone and which can be fixed to the retaining member and;
exerting a lateral clamping pressure directed towards the sagittal plane with the connecting elements.

41. The method of claim 40, wherein a control-element located in the retaining member coordinates the variation of the clamping pressure with each connecting element and each loop.

42. The method of claim 40, wherein each loop is supported on and led to at least two connecting elements.

43. The method of claim 40, wherein the loop, the connecting elements and the retaining member are composed of X-ray-transparent material.

44. The method of claim 40, wherein the loop, the connecting elements and the retaining member are composed of a material selected from the group consisting of light metals, light metal alloys, carbon and carbon-reinforced materials.

45. An external fixator for osteosynthesis, comprising:
an external retaining member configured and dimensioned for bridging separated bone segments;
a plurality of connecting elements configured and adapted for applying a clamping force to the separated bone segments, each connecting element having a first end and a second contact end, the first end configured and adapted for attachment to the retaining member and the second contact end configured and adapted for surface attachment to one of the separated bone segments without penetrating the bone segment;
a plurality of control elements configured and adapted for varying the clamping pressure applied by the connecting elements, at least one control element being coordinated with a connecting element and supported in the retaining member; and
a flexible loop connected to the retaining member, configured and adapted for exerting control tension on the connecting elements and of sufficient length to wrap around a medial and lateral surface of at least one bone segment.
